(19) United States
(12) Patent Application Publication
(10) Pub. No.: US 2004/0106997 A1
(43) Pub. Date: Jun. 3, 2004

(54) Apparatus and method for creating a surgical channel

(76) Inventor: Robert E. Lieberson, Pleasanton, CA (US)

Correspondence Address: KANG LIM, 3494 CAMINO TASSAJARA ROAD #436, DANVILLE, CA, 94306, US

(21) Appl. No.: 10/286,412

(22) Filed: Nov. 1, 2002

(51) Int. Cl. A61F 2/44

(52) U.S. Cl. 623/17.16; 606/61; 606/190

(57) ABSTRACT

A method and apparatus for creating safer and less intrusive surgical channels or pathways is provided. The dilatory apparatus includes a guide wire, two or more cannulated dilators, and a guide tube. The guide wire is inserted into the patient through a small skin incision. A cannulated dilator is inserted over the guide wire. Additional cannulated dilator(s) of increasing internal diameter(s) are inserted over the previously inserted dilator(s). When the desirable external diameter is reached, the guide tube is inserted over the outermost dilator. The dilators can now be removed to create a guided pathway formed by the guide tube. In one application, lumbar spinal surgery, a pair of diagonally aligned cavities is drilled in a spinal disc space using the guided pathways. A pair of vertebrae fusion cages can then be inserted the respective cavities, allowing the vertebrae to fuse together over time.

### BACKGROUND OF THE INVENTION

The present invention relates to the field of creating channels for surgery. More particularly, the present invention relates to less intrusive methods and apparatus for creating surgical channels during procedures such as spinal surgeries.

Chronic back pain is often caused by a herniated disc or a variety of degenerative spinal disc conditions where the anatomical function of the disc has been disrupted. The partially collapsed disc space causes adjacent vertebrae to pinch the nerves extending laterally from the spinal column resulting in chronic pain. The prevalent surgical treatment for correcting these types of conditions is to fuse the two vertebrae surrounding the affected disc.

In most cases, the affected disc is removed, and in order to prevent the collapse of the disc space, the excised disc space is stabilized by placing a parallel pair of cylindrical cages between the vertebrae as shown in FIGS. 1A and 1B. U.S. Pat. Nos. 6,371,986 and 6,375,655, herein incorporated by reference, are representative of the instrumentation, cages and procedures for vertebrae fusion.

There are generally two classes of procedures for accessing the spinal column to place the pair of cages. Both of these classes have serious disadvantages including associated risks of trauma to the surrounding tissue and extended recovery time.

As shown in FIG. 1A, the first class of procedures accesses the vertebrae from the anterior portion of the spinal column. These procedures include a fairly invasive incision through the patient's body, for example through the abdomen, to expose the anterior lumbar portion of the spinal column. The affected disc space 110 is distracted to a normal spacing and a pair parallel of cylindrical holes are drilled and tapped in disc space 110. Cages 122 and 124 are then inserted into the holes, enabling the vertebrae adjacent to disc space 110 to fuse with the cages over time.

Great care has to be taken when drilling and tapping via the anterior portion of the spinal column as major blood vessels are located close to the spinal column, including the vena cava and descending aorta, as well as the sympathetic nervous plexus. As discussed above, another major disadvantage is the need to penetrate the patient's body from the front thereby risking trauma to intervening organs and tissues.

FIG. 1B illustrates the second class of procedures which access the vertebrae from the posterior portion of the spinal column. Briefly, this procedure includes exposing the spinal column, and removing portions of the lamina 118 of both the upper and lower vertebrae adjacent to disc space 110. The spinal nerves can now be laterally displaced to access the posterior portion of disc space 110. A pair of holes is drilled and tapped into the distracted disc space 110. Cages 122 and 124 are then inserted in the holes.

Disadvantages of the posterior approach include the need to remove the protective lamina portion of the vertebrae, and the risk of trauma to the nerve roots and duramater.

Hence there is a need for a safer and less intrusive vertebrae fusion instrumentation and procedure that significantly reduces the risk of trauma to the surrounding tissues, thereby reducing the recovery time of the patient.

### SUMMARY OF THE INVENTION

To achieve the foregoing and in accordance with the present invention, a method and apparatus for creating safer and less intrusive surgical channels is provided. Such procedures reduce risk of trauma and recovery time for the patient.

In one embodiment, the dilatory apparatus includes a guide wire, two or more cannulated dilators, and a guide tube. The guide wire is inserted into the patient through a small skin incision. A cannulated dilator is inserted over the guide wire. Additional cannulated dilator(s) of increasing internal diameter(s) are inserted over the previously inserted dilator(s). When the desirable external diameter is reached, the guide tube is inserted over the outermost dilator. The dilators can now be removed to create a surgical pathway formed by the guide tube.

In one specific application, lumbar spinal surgery, a pair of diagonally aligned cavities are drilled in a spinal disc space using guided pathways created by the dilatory apparatus of the present invention. A pair of vertebrae fusion cages can then be inserted the respective cavities, allowing the vertebrae to fuse together over time.

In addition to spinal surgeries, the less intrusive channel or pathway may also be useful for other therapeutic and/or diagnostic procedures such as laparoscopy, retroperitoneal surgery and intraperitoneal surgery.

Note that the various features of the present invention, including the cannulated dilators and guide tube, can be practiced in combination with different surgical devices. These and other features of the present invention will be described in more detail below in the detailed description of the invention and in conjunction with the following figures.

### BRIEF DESCRIPTION OF THE DRAWINGS

The present invention is illustrated by way of example, and not by way of limitation, in the figures of the accompanying drawings and in which like reference numerals refer to similar elements and in which:

FIGS. 1A and 1B illustrate exemplary conventional anterior and posterior vertebrae fusion procedures, respectively.

FIG. 2 is a flow diagram illustrating the vertebrae procedure and apparatus of the present invention.

FIG. 3 is sectional view of a guide wire inserted into a disc space between adjacent vertebrae.

FIGS. 4A and 4D are sectional views showing the deployment of one embodiment of the serial dilators in accordance with the present invention.

FIG. 5 shows the deployment of a guide tube of the present invention over dilators of FIGS. 4A and 4D.

FIG. 6 shows the guide tube with the dilators removed.

FIGS. 7 and 8 show the drilling and tapping, respectively, of a disc space in accordance with the invention.

FIGS. 9 and 10 show the insertion of a cage diagonally from one side of the disc space.

FIG. 11 show a second cage inserted diagonally from the other side of the disc space.

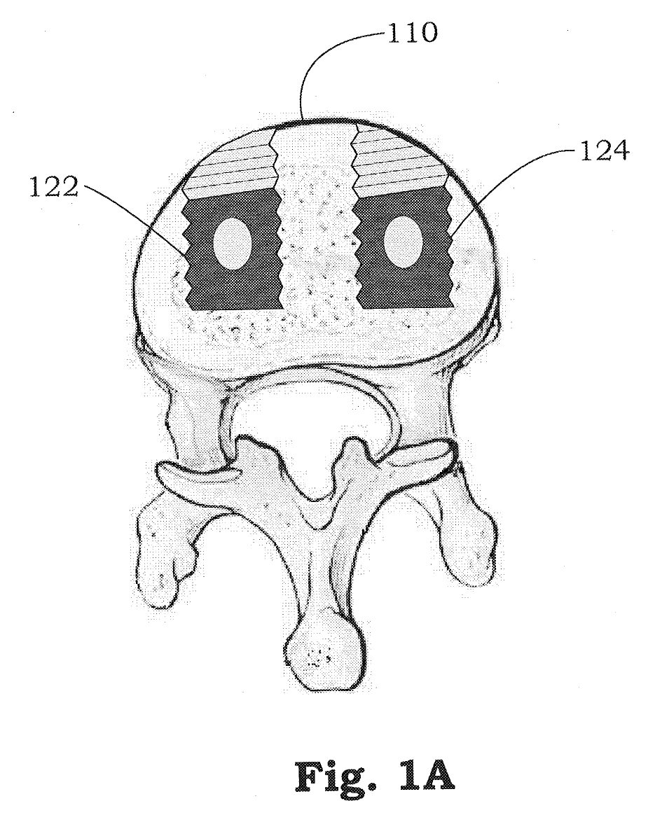

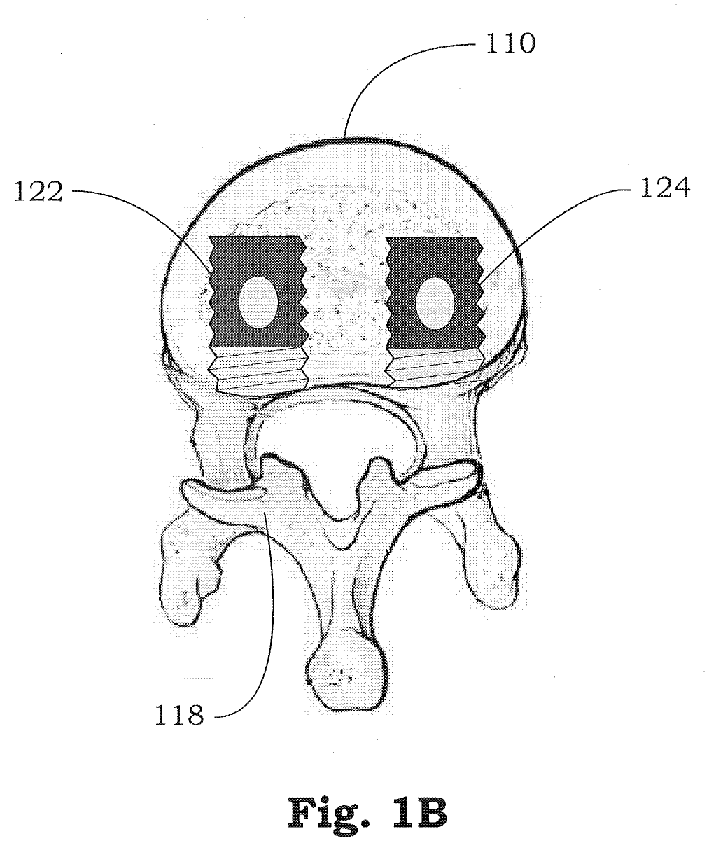

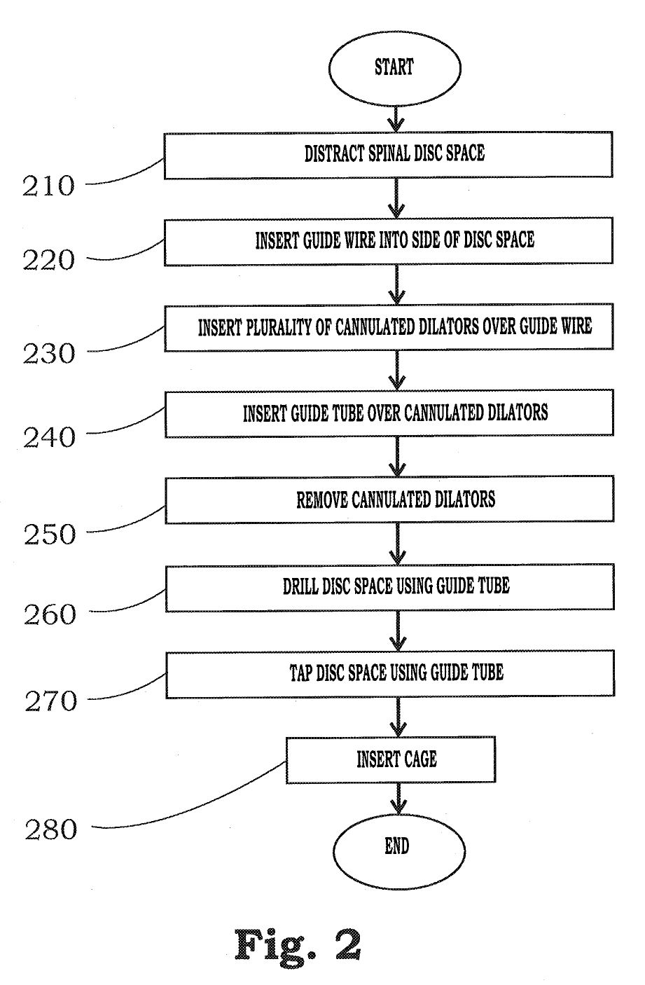

### DETAILED DESCRIPTION OF THE PREFERRED EMBODIMENTS

The present invention will now be described in detail with reference to a few preferred embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

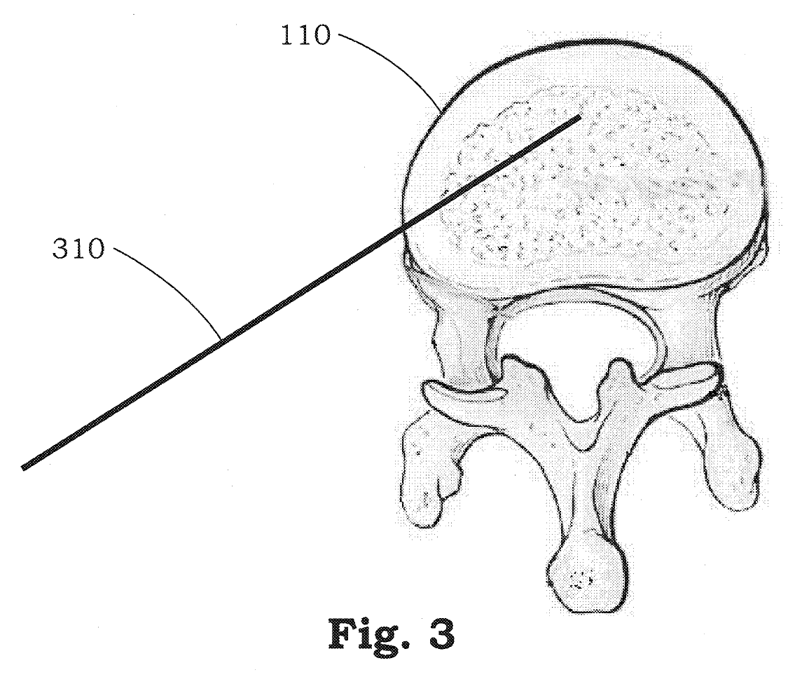

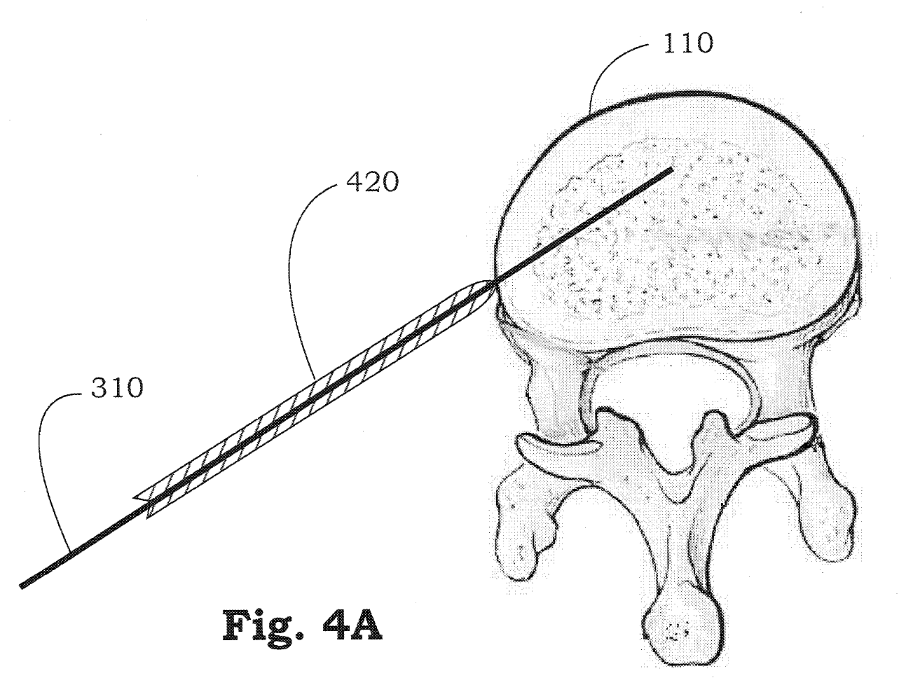

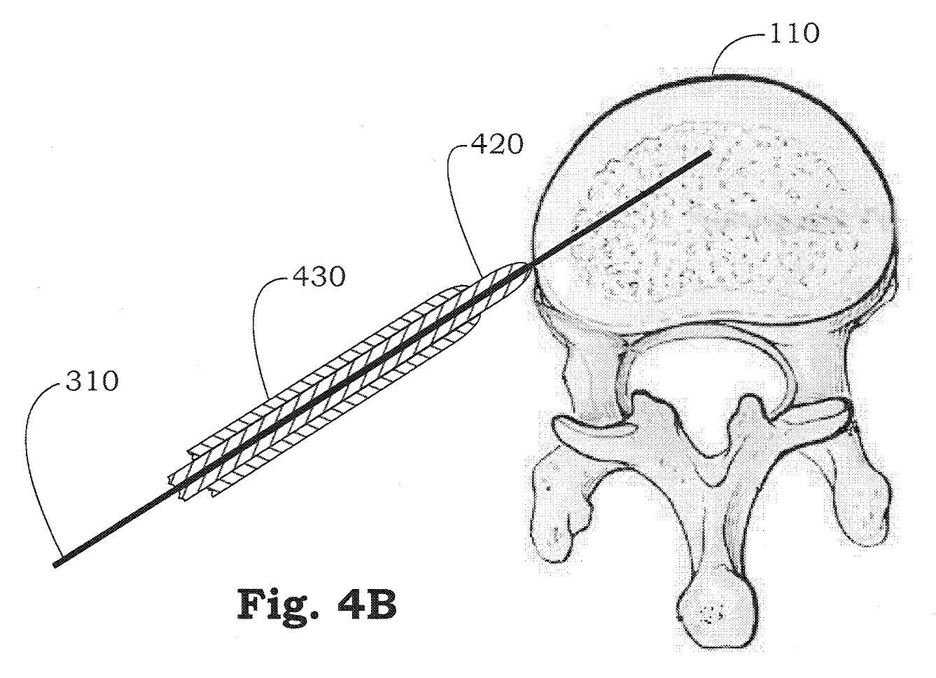

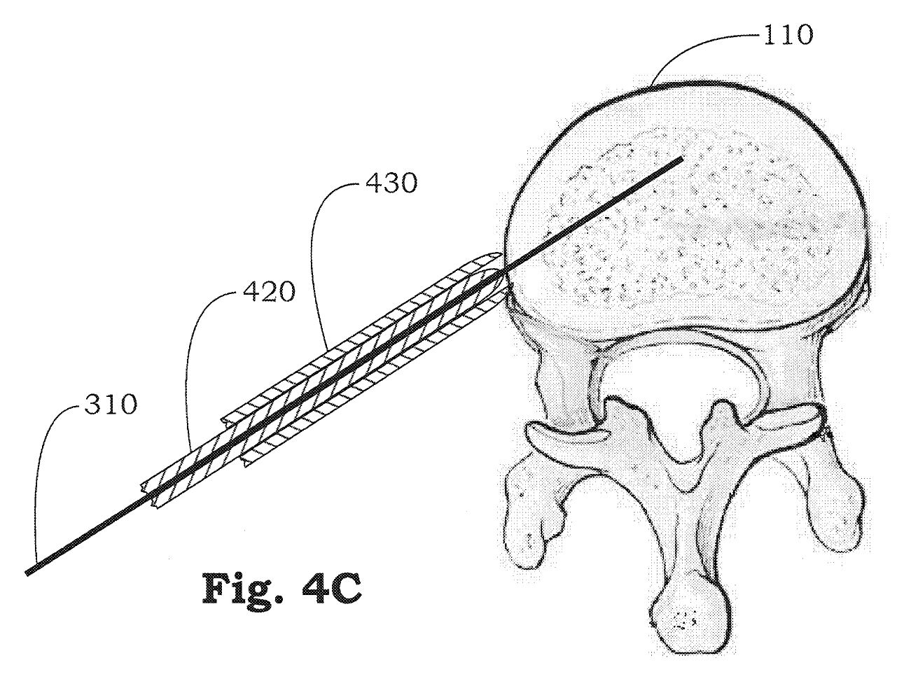

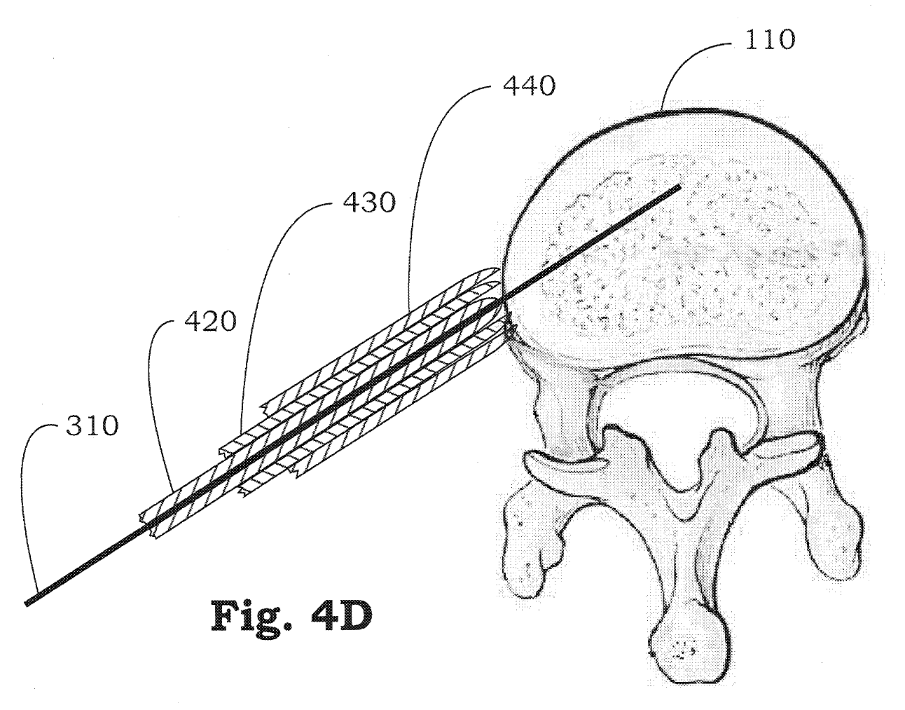

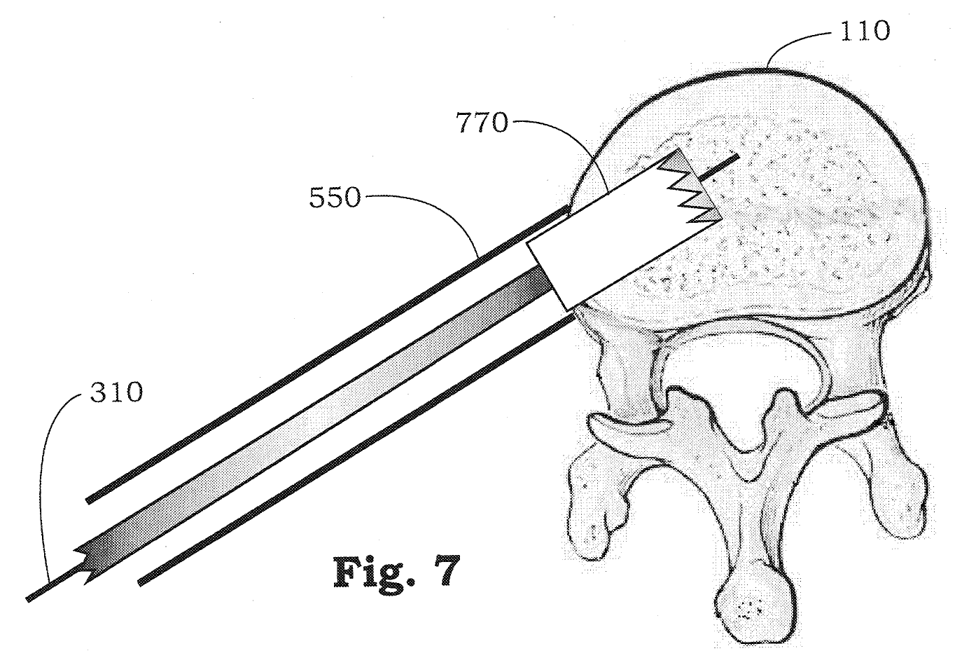

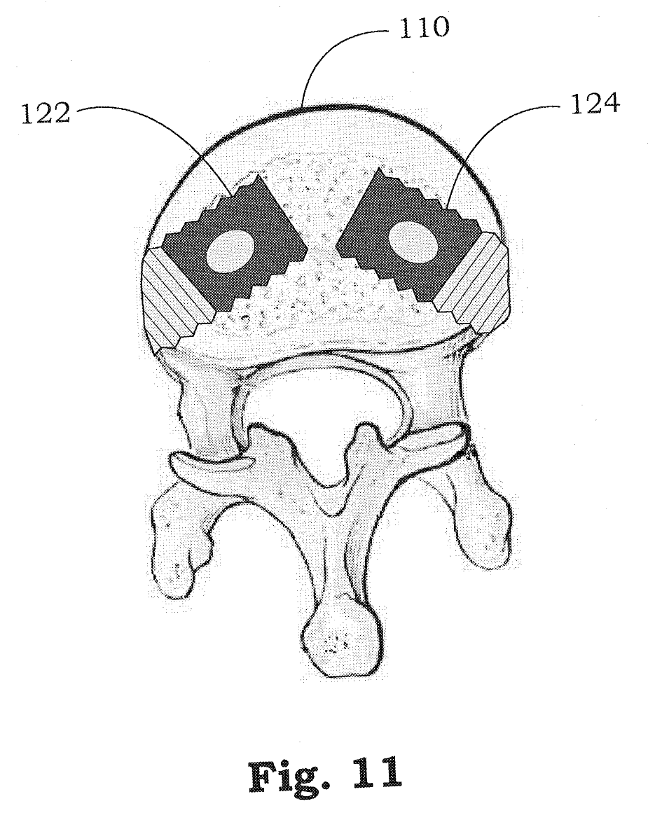

In accordance with the present invention, FIG. 2 is a flowchart illustrating a less invasive procedure for implanting vertebrae fusion cages into a disc space of a patient. FIG. 3 shows a guide wire inserted into the disc space. FIGS. 4A through 4D, 5 and 6 illustrate the operation of serial cannulated dilators and guide tube of the present invention. FIGS. 7 through 10 show the drilling, tapping and insertion of a cage diagonally into the disc space using the guide tube. FIG. 11 show both cages inserted into the disc space.

Depending on the degree of disc space failure, distraction of disc space 110 may be needed. One method of distraction includes inserting a pair of rigid metal pins (not shown), coupled together by to a lateral direct ion, into a corresponding pair of adjacent vertebrae. Pins can be formed from cylindrical stainless steel rods with a threaded leading end. Preferably, the pins are rigidly secured in parallel to each other and perpendicular to the spinal column.

A distraction tool (not shown), known to one skilled in the art, is used to provide distracting force between the pins, thereby returning disc space 110 to a normal spacing without introducing any relative rotation between the vertebrae (step 210). U.S. Pat. No. 4,898,161 teaches a suitable distracting tool and is herein incorporated by reference.

Referring now to FIG. 3, using a fluoroscope (not shown), a guide wire 310 is inserted diagonally into disc space 110 for a depth of approximately 15 to 25 mm (step 220). This depth is preferable because guide wire 310 functions as a guide rod in a manner described below. Guide wire 310 can be a Kirschner wire (K-wire) available from SSR/Miltex of Oyster Bay, N.Y.

FIG. 4A shows a first cannulated dilator 420 being inserted into the patient using guide wire 310 as a guide. As first cannulated dilator 420 is inserted over guide wire 310, the surrounding tissue is distended to the diameter of dilator 420. As shown in FIGS. 4B and 4C, a second cannulated dilator 430 is then inserted over first dilator 420, further distending the surrounding tissue. In FIG. 4D, a third cannulated dilator 440 is inserted over second cannulated dilator 430. See step 230. In order to avoid tearing the skin, a skin incision of about 20 mm is made prior to the insert of dilators 420, 430 and 440.

While three serial cannulated dilators are described in this embodiment, other embodiments which include one or more cannulated dilators are possible. In addition, depending on the surgical procedure, it is also contemplated that the cannulated dilators have varying wall thickness and a variety of lengths. For example in spinal surgery, the wall thickness may range from 2 to 5 mm and the length may range from 100 to 200 mm. Suitable materials for cannulated dilators 420, 430 and 440 include metals, plastics, and ceramics known to one skilled in the surgical instrumentation art, such as titanium or hardened aluminum.

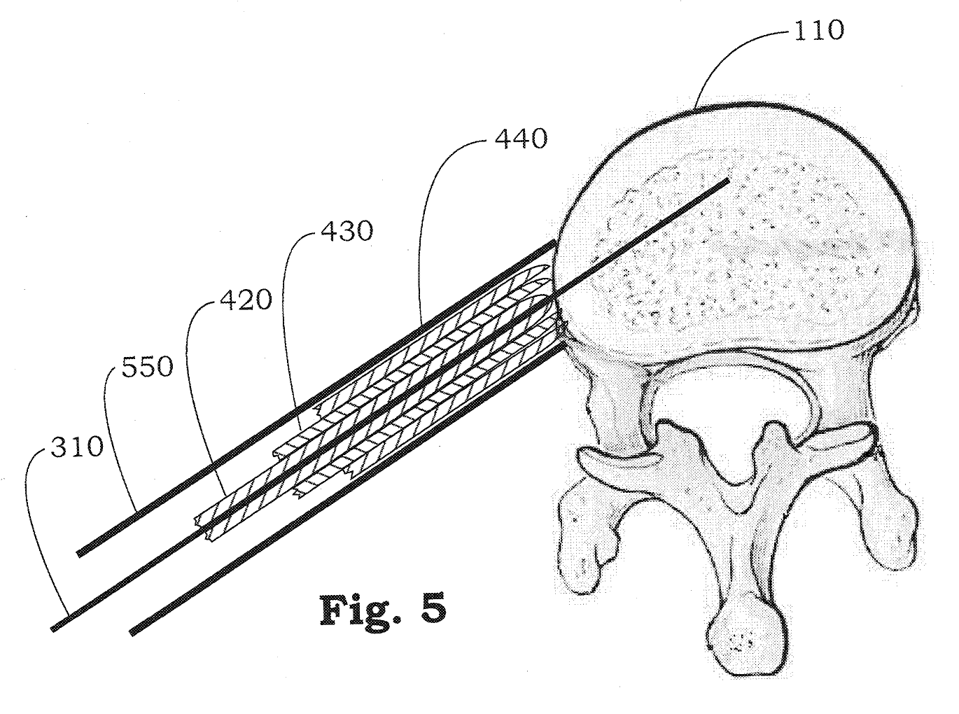

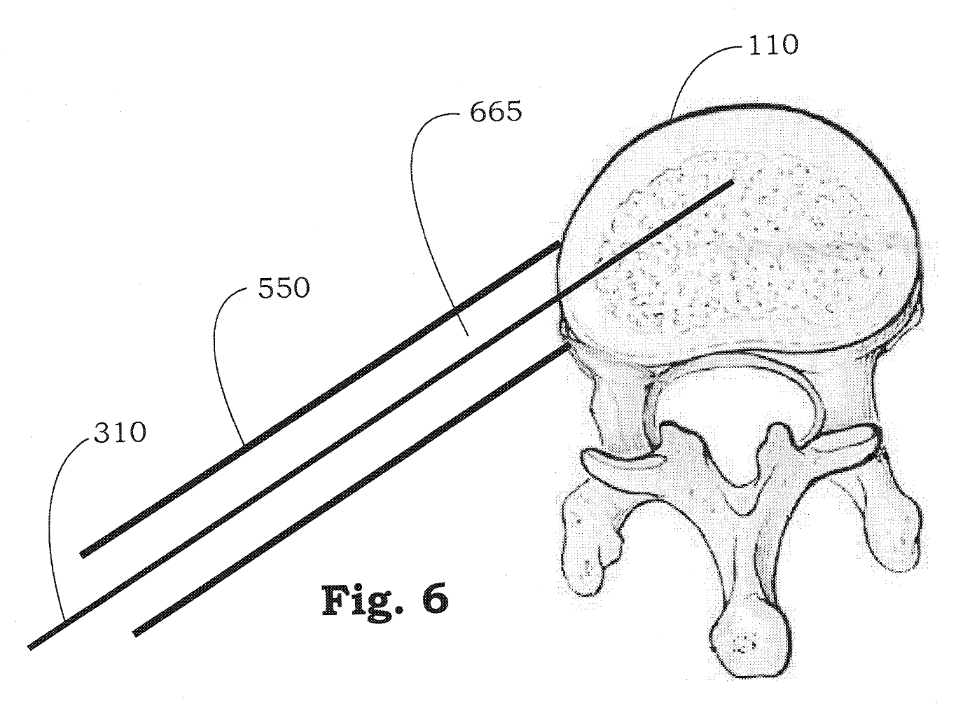

Next, as illustrated by FIGS. 5 and 6, a guide tube 550 is inserted over the plurality of dilators 420, 430 and 440, which can then withdraw leaving a surgical channel 665 within guide tube 550 (steps 240 and 250). In this embodiment, guide tube is about 16 to 20 mm in diameter and 100 to 150 mm in length. The wall of guide tube 550 is approximately 1 mm. Suitable materials for guide tube 550 include metals, plastics and ceramics used for surgical instruments, such as stainless steel, titanium, and aluminum.

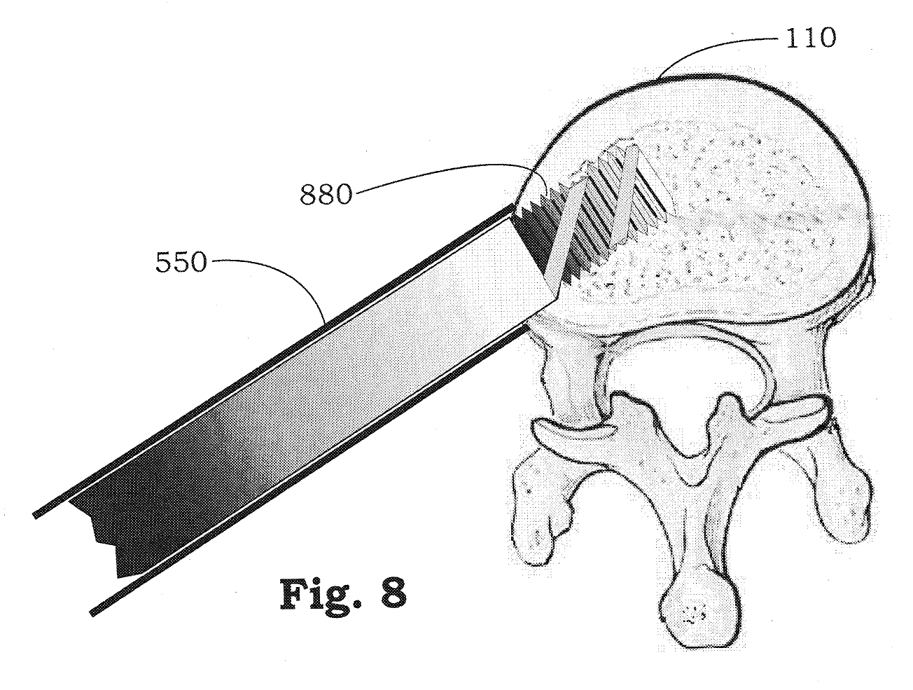

In one application of the present invention involving vertebrae fusion surgery as shown in FIG. 7, a hole is drilled into disc space 110 via surgical channel 665 (step 260). Guide tube 550 and K-wire 310 function together to protect the surrounding tissues while guiding the drill into disc space 110. FIG. 8 shows a tap 880 with its threading head in disc space 110 and its tool body in guide tube 550 (step 270). Drills, drill bits and taps are commercially available from Stryker Corporation of Kalamazoo, Mich., and Medtronic Sofamor-Danek of Memphis, Tenn.

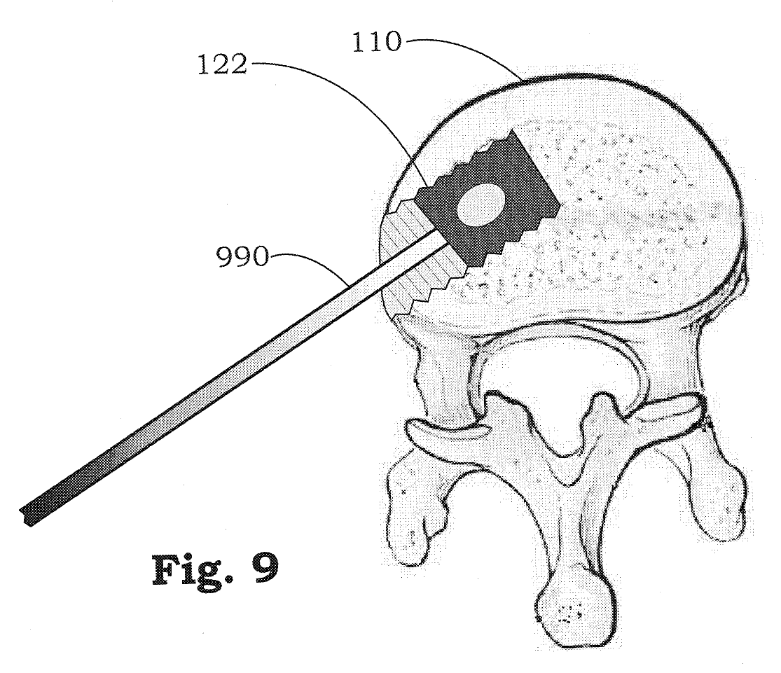

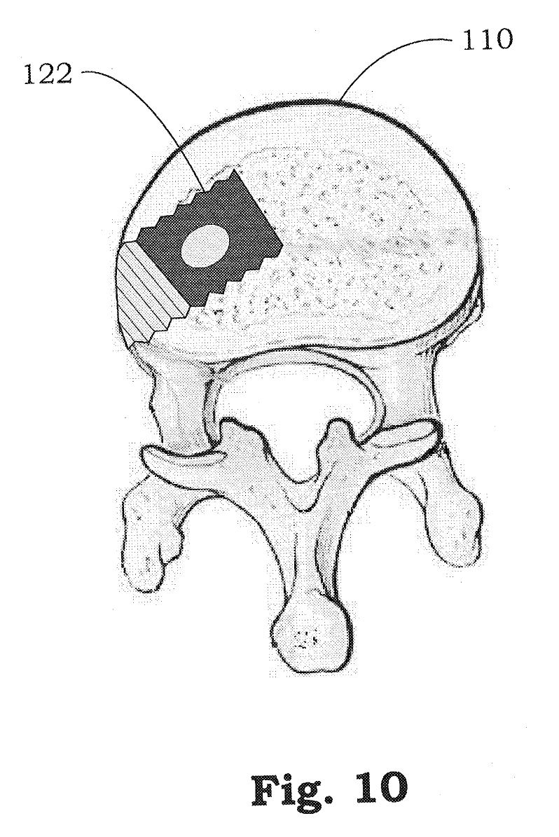

Referring to FIGS. 9 and 10, a fusion cage 122 is inserted into the tapped hole in disc space 110 using an insertion tool 990 (step 280). There are several fusion cages available commercially, including BAKTM cage from Spine-Tech of Minneapolis, Minnesota, and LT-CAGE™ and INTER FIX™ cages from Medtronic Sofamor-Danek of Memphis, Tenn.

As illustrated by FIG. 11, steps 220 through 280 are repeated to insert a second fusion cage 124 diagonally opposed to the first fusion cage 122.

The advantages of inserting cages 122 and 124 diagonally include minimal invasive access to the disk space, avoiding lamina removal, and avoiding surgery close the spinal cord and major blood vessel. Hence, recovery time and the risk of complications are both also greatly decreased, since there is a smaller incision, less blood loss, lower risk in infection and less pain.

Many variations of the present invention are possible. For example guide tube 550 may have a rough leading edge so that it can secure the vertebrae adjacent to disc space 110, thereby increasing the stability of surgical channel 665. Drilling and tapping of disc space 110 may be accomplished without guide wire 310.

Depending on the application, it may also be possible to use dilators 420, 430 and 440 without guide wire 310. In some applications, a relatively small surgical channel 665 in needed, and a single dilator 420 may be sufficient. While guide tube 550 is preferably thinner than dilators 420, 430 and 440, conceptually, guide tube 550 is a thin-walled outmost dilator.

Surgical channel 665 can be used with different types of diagnostic and surgical procedures. Exemplary diagnostic and therapeutic applications for surgical channel 665 include placement of internal distractor for correction of scotiosis, retroperitoneal surgery and intraperitoneal surgery.

While this invention has been described in terms of several preferred embodiments, there are alterations, modifications, permutations, and substitute equivalents, which fall within the scope of this invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method of surgically creating a guided pathway in a patient, the method comprising:
inserting a guide wire into the patient;
inserting at least two cannulated dilators over the guide wire;
inserting a guide tube over the dilators; and
removing the dilators, thereby creating the guided pathway.

2. The method of claim 1 further comprising:
creating a cavity into a vertebrae disc space via the guided pathway; and
inserting a vertebrae fusion cage into the cavity.

3. The method of claim 2 further comprising:
creating a second cavity into the vertebrae disc space via a second guided pathway, said second cavity diagonally aligned with respect to said cavity; and
inserting a second vertebrae fusion cage into the second cavity.

4. A method of surgically creating a guided pathway in a patient, the method comprising:
inserting at least one dilator into the patient;
inserting a guide tube over the at least one dilator; and
removing the at least one dilator, thereby creating the guided pathway.

5. The method of claim 1 wherein the at least one dilator is cannulated and a guide wire is inserted into the patient before the at least one dilator is inserted over the guide wire.

6. A dilatory apparatus useful for creating a surgical pathway in a patient, the dilatory apparatus comprising:
a guide wire;
a first cannulated dilator configured to slide over the guide wire;
a second cannulated dilator configured to slide over the first cannulated dilator; and
a guide tube configured to slide over the second cannulated dilator, and wherein the first and second cannulated dilators can be removed to form the surgical pathway.

7. A dilatory apparatus useful for creating a surgical pathway in a patient, the dilatory apparatus comprising:
at least one dilator; and
a guide tube configured to slide over the at least one dilator which can be removed to form the surgical pathway.

8. The apparatus of claim 7 further comprises a guide wire, and wherein the at least one dilator is cannulated and configured to slide over the guide wire.
